(19) United States
(12) Patent Application Publication
(10) Pub. No.: US 2003/0173110 A1
(43) Pub. Date: Sep. 18, 2003

(54) Electrically-mediated transdermal drug injection

(76) Inventors: Yosef Gross, Moshav Mazor (IL); Zohar Avrahami, Rehovot (IL)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/275,531

(22) PCT Filed: May 3, 2001
(86) PCT No.: PCT/IL01/00397

(30) Foreign Application Priority Data
May 7, 2000 (IL) 136008

(51) Int. Cl. H05K 1/16

(52) U.S. Cl. 174/260

(57) ABSTRACT

Apparatus (20) for injection of a substance (28) through the stratum corneum of skin (26) is provided. The apparatus includes a reservoir (42), containing the substance. The reservoir preferably has at least one outlet. A pressure generator (22) applies a pressure to eject the substance through the at least one outlet. One or more electrodes (32, 34) in a vicinity of the outlet are placed on the skin. Energy is conveyed through the electrodes to the skin, to facilitate passage of the substance through the stratum corneum.

### FIELD OF THE INVENTION

The present invention relates generally to drug delivery devices and methods, and specifically to electrically-mediated transdermal drug delivery.

### BACKGROUND OF THE INVENTION

Methods for delivering a drug through a patient's skin are well known in the art, and include passive diffusion of the drug from a skin patch to the skin, and active processes such as hypodermic injection, iontophoresis, sonophoresis, electroporation, laser ablation, and chemically-enhanced diffusion. Each of these methods is typically limited by one or more of the following:

a needle and/or tissue-heating causes the patient pain,

tissue-heating causes unnecessary damage,

generation of a hole in the skin and transfer of the drug are performed in two separate steps,

expensive apparatus is required,

only relatively small molecules are conveyed, and

the rate of drug transfer is low.

For example, U.S. Pat. Nos. 4,775,361, 5,165,418, and 5,423,803, and PCT Publication WO 97/07734, which are incorporated herein by reference, describe methods of using laser pulses to locally heat the stratum corneum to about 120° C., thereby causing local ablation, in order to cause a single hole to develop in the stratum corneum through which large molecules may pass. PCT Publication WO 97/07734 also discloses thermal ablation of the stratum corneum using an electrically-resistive element in contact with the stratum corneum, such that a high current through the element causes a general heating of tissue in its vicinity, most particularly the stratum corneum. Electroporation is well known in the art and is described, for example, in an article by Chizmadzhev et al., entitled, “Electrical properties of skin at moderate voltages,” Biophysics Journal, February, 1998, 74(2), pp. 843-856, and in U.S. Pat. No. 5,019,034, both of which are incorporated herein by reference. All of these methods are characterized by at least one of the above-listed limitations.

U.S. Pat. No. 5,304,128 to Haber et al., which is incorporated herein by reference, describes a syringe that includes a gas-driven piston to force liquid medication from the syringe through an injection nozzle. Pressure-driven devices of this sort obviate the need for a hypodermic needle, and they are therefore frequently called “needle-less injectors.” Such devices must be able to produce and withstand very high gas pressure, which is needed to drive the medication through the stratum corneum. For this reason, such devices are costly and cannot practically be made as disposable products.

### SUMMARY OF THE INVENTION

It is an object of some aspects of the present invention to provide improved apparatus and methods for transdermal injection of a substance.

It is an additional object of some aspects of the present invention to provide improved apparatus and methods for simplifying the process of transdermal injection of a substance.

It is a further object of some aspects of the present invention to provide improved apparatus and methods for increasing the speed of transdermal delivery of a substance.

It is yet a further object of some aspects of the present invention to provide improved apparatus and methods for increasing the cost-efficiency of transdermal injection of a substance.

In preferred embodiments of the present invention, an electrically-assisted injection device injects a substance through the stratum corneum of a patient's skin. The device comprises a reservoir, containing the substance, and a pressure generator, which applies a pressure to eject the substance from an outlet of the reservoir. Additionally, the device comprises at least two electrodes in a vicinity of the outlet. The electrodes are placed on the patient's skin, and electrical energy is conveyed through the electrodes to the skin in order to facilitate passage of the substance through the stratum corneum.

Preferably—and unlike comparable drug-delivery systems known in the art—the substance is delivered to the patient in a painless manner, without the use of needles. The electrically-assisted injection device is preferably an inexpensive, single-use device, which requires no special skill to operate, and which administers the substance responsive to a single action by a nurse or the patient.

In some preferred embodiments of the present invention, parts of the device are constructed in a manner generally similar to a syringe, whereby a plunger, coupled to move within a barrel, generates the pressure in order to eject the substance. In these embodiments, the reservoir is preferably within the barrel, and the outlet comprises the nozzle of the syringe. Typically, the syringe is provided pre-filled with the substance, and the plunger is in a retracted position thereof When the patient or the nurse applies a compressive force to the plunger, the electrodes are actuated, and the substance is ejected immediately thereafter. Alternatively, the syringe is a general-purpose drug-delivery device, which the nurse loads with a desired substance prior to administration.

For some applications, it is desirable to provide pressure in support or in place of that generated by manual compression of the plunger. This may be particularly useful when a relatively large quantity of the substance is to be delivered through the stratum corneum, as facilitated by the electrodes, to be passed deep into the dermis or into the bloodstream. In these cases, therefore, pressure is preferably generated by active methods, for example, one or more of the following:

electrostatic force, generated within the barrel, which accelerates the substance to pass through the nozzle at high velocity,

a small explosion, which creates a shock-wave in the barrel, to propel the substance at high velocity out of the syringe,

a pre-tensed spring, which pulls or pushes the plunger, in order to drive the substance out of the barrel, and

an electrolytic reaction, which rapidly increases the air-pressure in the barrel, driving out liquid substance.

Preferably, compression of the plunger actuates the electrodes to convey current to the patient's skin. In particular, movement of the plunger from the retracted position towards a compressed position thereof preferably closes an electric circuit, such that the current is enabled to flow from a charge-storage element, such as a capacitor or battery, into the patient's skin. Typically, the charge-storage element (and the syringe as a whole) is a single-use item, and is provided with sufficient charge stored therein to facilitate the flow of the substance, as provided by embodiments of the present invention. Alternatively, the syringe is designed for multiple administrations of one or more substances, and the charge-storage element is replaceable or rechargeable.

Preferably, use of a charge-storage element as described herein defines a maximum quantity of charge that may flow through the electrodes. Therefore, the element may be used to reduce or eliminate the possibility of undesired injury to the skin responsive to the passage of current therethrough. In some embodiments, the charge-storage element comprises resistors and other passive or active elements, which modify aspects of the current flow.

U.S. patent application Ser. No. 09/189,170, filed Nov. 9, 1998, entitled, “Transdermal drug delivery and analyte extraction,” which is assigned to the assignee of the present patent application and is incorporated herein by reference, describes a device for enhancing transdermal movement of a substance. The device includes: (a) a skin patch, with at least two electrodes in contact with a subject's skin; and (b) a control unit, coupled to the patch, which causes a current to pass between the electrodes through the stratum corneum. Application of the current causes micro-channels to form in the stratum corneum to enable or augment transdermal movement of the substance. The control unit typically has switching circuitry to control the magnitude and/or duration of the electric field at the electrodes.

In some preferred embodiments of the present invention, the current flow generates micro-channels in the patient's skin, and thereby facilitates the desired passage of the substance through the skin. Micro-channel generation as practiced in these embodiments typically uses methods such as are described in the above-mentioned U.S. patent application Ser. No. 09/189,170. The term “micro-channel,” as used in the context of the present patent application, refers to a pathway generally extending from the surface of the skin through all or a significant part of the stratum corneum, through which pathway molecules can diffuse. Preferably, micro-channels allow the diffusion therethrough of large molecules at a greater rate than the same molecules would diffuse through pores generated by electroporation. The combination of such micro-channels with pressure-driven drug injection enables a far larger quantity of the medication to penetrate through the skin in a short time that would otherwise be possible.

Generally, the current flow between the electrodes can be described as having two components: (a) a perpendicular component, generally perpendicular to the skin surface; and (b) a lateral component, generally parallel to the skin surface. If the perpendicular component is too large, it may cause current to go through the stratum corneum into the underlying innervated, pain-sensitive, epidermal tissue and dermis.

Therefore, in embodiments of the present invention wherein micro-channels are generated, methods and/or apparatus are preferably employed to increase the relative value of the lateral component with respect to the perpendicular component. In general, the stratum corneum demonstrates a significantly higher resistance to the passage of molecules therethrough than does the underlying epidermal tissue. It is therefore an object of these embodiments to form micro-channels in the stratum corneum by ablating the stratum corneum, in order to increase conductance of the substance therethrough, generally without directly affecting or damaging epidermal tissue underlying the stratum corneum. Limiting current flow substantially to the non-innervated stratum corneum is believed to decrease or eliminate the patient's sensations, discomfort, or pain responsive to use of these embodiments of the present invention, particularly as compared with other injection procedures known in the art.

Alternatively or additionally, other electrically-mediated transdermal drug-delivery modalities known in the art are utilized to facilitate delivery of the substance, typically by decreasing resistance of the stratum corneum to the passage therethrough of the substance.

In some preferred embodiments of the present invention, an array of electrodes is deployed around the outlet, or around multiple outlets of the device, in order to further increase the transfer rate of the substance into the skin. Preferably, the array comprises closely-spaced electrodes, which generally act together to produce a high micro-channel density in an affected area of the skin. Alternatively or additionally, the array of electrodes conveys the current using other methods known in the art, in order to ablate or otherwise modify the stratum corneum, and thereby facilitate passage of the substance through the stratum corneum.

The present invention will be more fully understood from the following detailed description of the preferred embodiments thereof, taken together with the drawings, in which:

### BRIEF DESCRIPTION OF THE DRAWINGS

FIGS. 1A and 1B are schematic, sectional illustrations of a device for electrically-mediated transdermal injection of a substance, in respective retracted and compressed positions thereof, in accordance with a preferred embodiment of the present invention;

FIG. 2 is a schematic, pictorial illustration of the device of FIG. 1A;

FIG. 3 is a schematic, pictorial illustration of another transdermal delivery device, in accordance with a preferred embodiment of the present invention; and

FIGS. 4, 5, and 6 are schematic, sectional illustrations of still other transdermal injection devices, in accordance with preferred embodiments of the present invention.

### DETAILED DESCRIPTION OF PREFERRED EMBODIMENTS

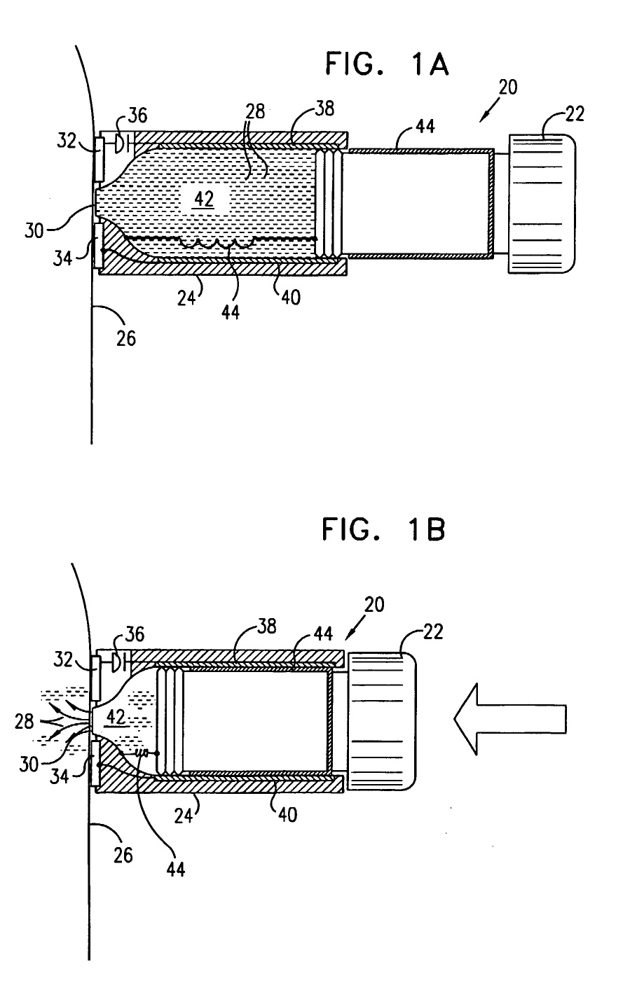

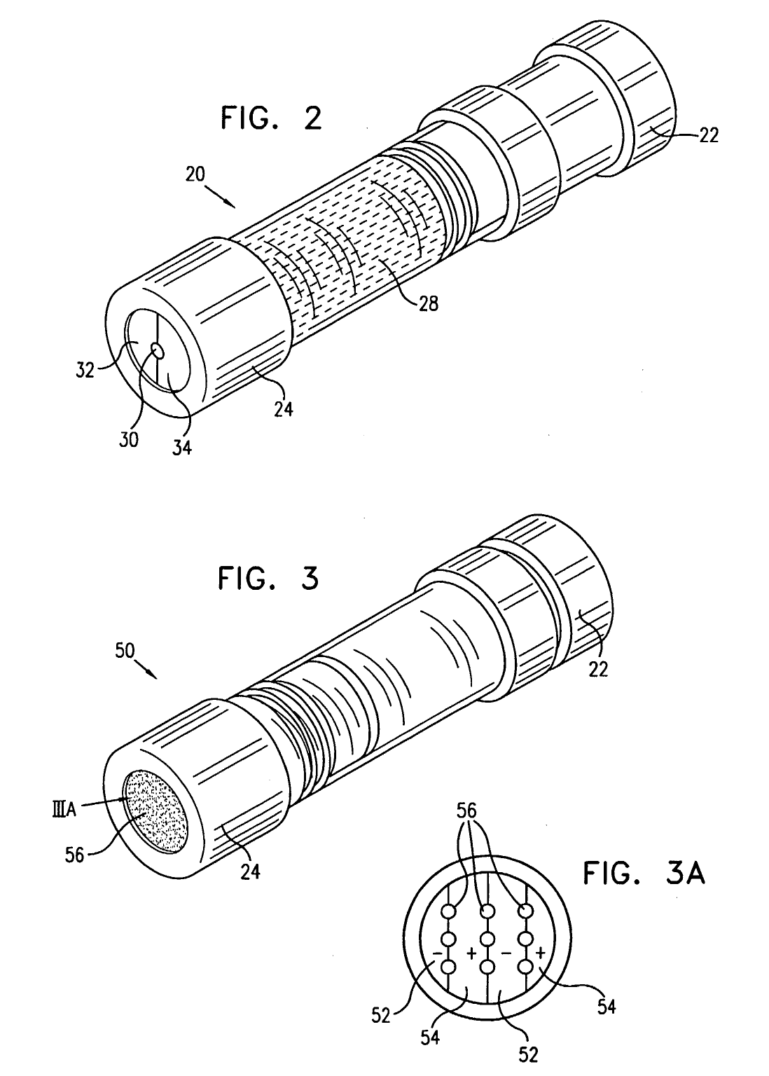

Reference is now made to FIGS. 1A, 1B, and 2. FIGS. 1A and 1B are schematic, sectional illustrations of a device 20 for delivery of a substance 28 into the skin 26 of a patient, in accordance with a preferred embodiment of the present invention. FIG. 2 is a schematic, pictorial illustration of device 20. Device 20 preferably has a form generally similar to that of a syringe, and is described herein with respect to two positions thereof: a retracted position, as shown in FIG. 1A, and a compressed position, as shown in FIG. 1B. In the retracted position, a plunger 22 is withdrawn from a barrel 24 of device 20. Movement of plunger 22 through the barrel into the compressed position preferably creates a pressure on a reservoir 42 containing substance 28, driving the substance out of the reservoir through an outlet 30, and into skin 26.

Preferably, device 20 comprises two electrodes 32 and 34, in a vicinity of outlet 30, which are placed on skin 26 prior to operating device 20. On actuation, electrical energy is conveyed through the electrodes to the skin. Application of electrical to the skin in this manner, as described further hereinbelow, facilitates passage of substance 28 through the stratum corneum of skin 26.

Preferably, compression of plunger 22 actuates the electrodes to convey current to the patient's skin. As shown in FIGS. 1A and 1B, movement of the plunger from the retracted position towards the compressed position closes an electric circuit, by bringing a sliding, electrically-conductive member 44 into electrical contact with fixed electrically-conductive members 38 and 40, such that the current is enabled to flow from a charge-storage element 36 coupled to electrode 32 into the patient's skin.

Typically, charge-storage element 36 is designed for single-use, and comprises a capacitor (most preferably a super-capacitor, as is known in the art) or battery that is provided with sufficient charge stored therein to facilitate the delivery of the substance, as provided by embodiments of the present invention. Alternatively, device 20, including element 36, is designed for multiple administrations of one or more substances, and the charge-storage element is replaceable or rechargeable. For applications in which device 20 is required to have a long shelf-life, it is preferable to enable the device to be charged shortly before use, typically by exposing electrodes 32 and 34 and applying a voltage therebetween.

Because charge-storage element 36 is typically not connected to an external source of energy during operation of device 20, it generally can only release a quantity of charge through the electrodes that is less than or equal to the total charge stored in the element. For some applications, the total stored charge is regulated to reduce or eliminate the possibility of pain and/or undesired injury to the skin responsive to the passage of current therethrough.

In some embodiments, the charge-storage element comprises resistors and other passive or active elements (not shown in the figures), which modify aspects of the current flow to mitigate pain or injury. Although preferred embodiments of the present invention are described herein with respect to applying substantially constant current or voltage through electrodes 32 and 34, it is within the scope of the invention to apply more complex electric fields, for example, including alternating current components, square pulses, etc.

Preferably, the current flow from electrodes 32 and 34 generates micro-channels in skin 26, and thereby facilitates the desired passage of substance 28 through the skin. Micro-channel generation typically uses methods similar to those described in the above-cited U.S. patent application, “Transdermal drug delivery and analyte extraction.” In particular, apparatus and methods (e.g., stimulation parameters and electrode-placement parameters) described in that application are preferably used in some applications of the present invention to ablate a local region of the stratum corneum, in order to increase conductance of the substance therethrough, generally without damaging epidermal tissue underlying the stratum corneum. Limiting current flow substantially to the non-innervated stratum corneum is believed to decrease or eliminate the patient's sensations, discomfort, or pain responsive to use of these embodiments of the present invention.

Alternatively or additionally, one or more other electrically-mediated transdermal drug-delivery procedures known in the art, including, for example, electroporation or iontophoresis, are utilized to enhance the passage of substance 28 into the skin.

An optional mechanical-energy storage element 44, e.g., comprising a spring and/or a compressed-gas container, is coupled between plunger 22 and barrel 24. For applications in which element 44 comprises a spring, the spring is held in tension (or compression) when device 20 is in the retracted position. A locking mechanism (not shown) preferably prevents the spring from contracting, typically until plunger 22 has compressed a predetermined distance. The force which expels substance 28 from outlet 30 is preferably supplemented by the release of energy associated with the return of the spring to a neutral position thereof.

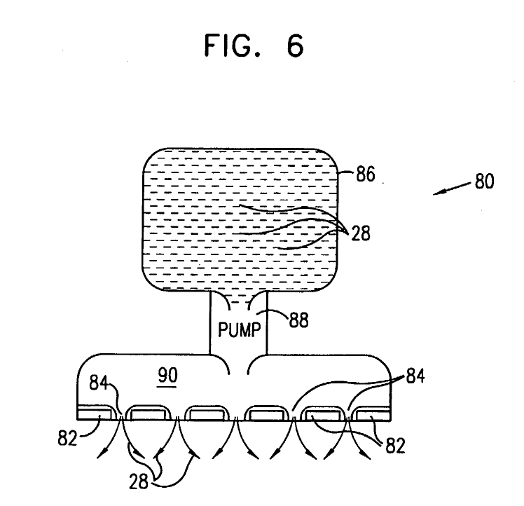

Typically, substance 28 comprises a therapeutic pharmaceutical product, such as a vaccine, or a diagnosis-related product, e.g., a radioactive compound. For most applications, substance 28 can be in a liquid or particulate form, although for particular embodiments of the present invention (for example, that described hereinbelow with reference to FIG. 4), one or the other form is preferred.

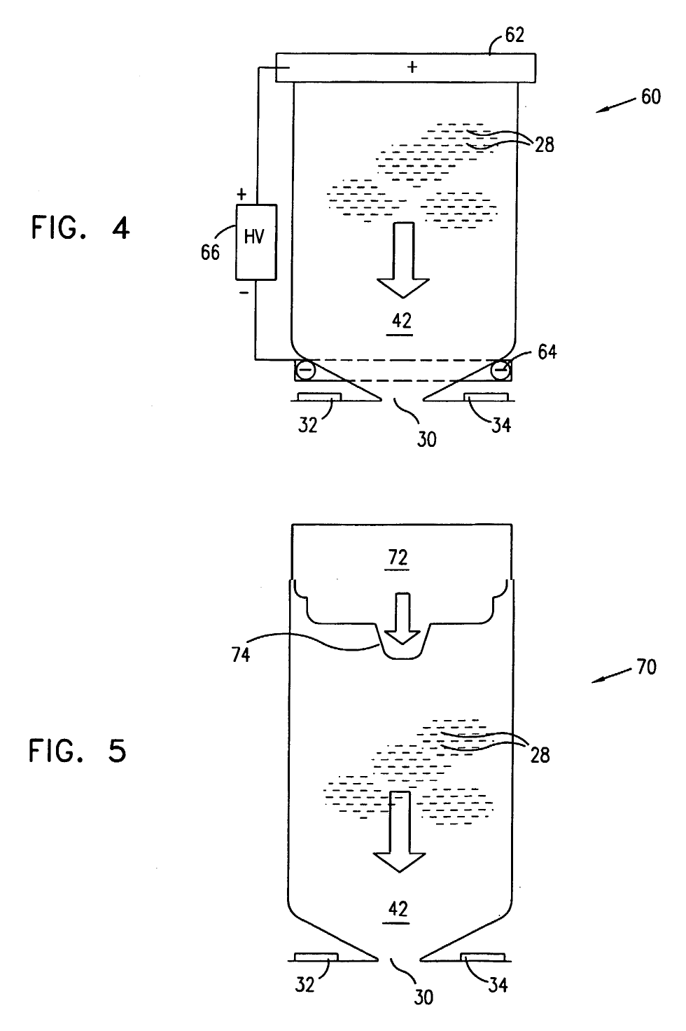

FIG. 3 is a schematic, pictorial illustration of another device 50 for transdermal delivery of substance 28, in accordance with a preferred embodiment of the present invention. Device 50 is similar in many respects to device 20, described hereinabove, differing generally only in the placement of electrodes and outlets. Preferably, device 50 comprises an array of negative and positive electrodes 52 and 54 disposed near a set of outlets 56 of reservoir 42. The electrode array may be linear, as shown in FIG. 3, or, for example, may comprise a grid of electrodes (not shown). When actuated, the electrodes generate an electric field, which ablates the stratum corneum or otherwise modifies properties of skin 26, in order to facilitate delivery of the substance into the patient's skin. Generally, placement of a plurality of positive and negative electrodes on skin 26, and/or the use of a plurality of reservoir-outlets, increases the rate of transfer of the substance into the patient's skin.

Reference is now made to FIGS. 1A, 1B, 4, 5, and 6. For some applications, it is desirable to provide pressure in support or in place of that generated by manual compression of plunger 22. This may be particularly useful, for example, when a relatively large quantity of substance 28 is to be delivered through the stratum corneum, and then deep into the dermis, or into the bloodstream. In these cases, therefore, additional pressure is preferably generated by active methods, as described herein.

FIG. 4 is a schematic, sectional illustration of yet another device 60 for transdermal delivery of substance 28, in accordance with a preferred embodiment of the present invention. Device 60 is generally similar to device 20, described hereinabove with reference to FIG. 1A, but is distinguished therefrom by the use of a high voltage source 66, which is actuated by an operator of device 60 to apply a voltage between an upper surface 62 and a ring electrode 64, generally surrounding outlet 30. Preferably, substance 28 comprises powder particles, the particles in turn comprising an insulating, charge-carrying material. Preferably, although not necessarily, the polarity between surface 62 and ring electrode 64 is alternated several times prior to administration of the substance in order to de-aggregate the powder particles. Thereafter, the electric field is generated between electrodes 32 and 34, in order to modify a property of the skin, e.g., to ablate the stratum corneum, and source 66 applies a voltage between electrodes 62 and 64, in order to accelerate the charged particles of substance 28 into the affected area of skin 26. The movement of substance 28 is optionally enhanced by manually compressing plunger 22 (not shown in this figure).

Some appropriate methods for generating the high voltage, for de-aggregating the powder, and for administering drugs in the powder form are described in U.S. patent application Ser. No. 09/326,111, entitled, “Powder inhaler,” which is assigned to the assignee of the present patent application and is incorporated herein by reference. In that application, a method is described for delivery of a dry powder to a patient by inhalation. The powder is de-aggregated and mobilized by application of a magnetic field. The field interacts with the package, engendering rapid motion thereof, which de-aggregates the powder.

Alternatively or additionally, useful techniques for applying electric fields to powders and managing drugs in the powder form, which may be utilized in some embodiments of the present invention, are described in U.S. Pat. No. 5,983,135, entitled, “Transdermal delivery of fine powders,” which is incorporated herein by reference. In that application, a powder delivery patch is described, including an electrostatic pad and an electrical power source. In preparation for application of a dry powder, such as a drug in powder form, to the skin of a subject, the power source applies an electrical potential to the pad, which causes the powder to adhere by electrostatic force to a lower side of the pad. This side is placed against the skin, and the electrical potential on the pad is reversed. The resultant electrostatic force drives the powder off the pad and onto the skin, through which the powder is absorbed into the body.

FIG. 5 is a schematic, sectional illustration of still another device 70 for transdermal delivery of substance 28, in accordance with a preferred embodiment of the present invention. Preferably, a small explosion is generated in a region 72 of device 70, such that a shock-wave 74 created by the explosion causes or enhances the flow of substance 28 through outlet 30 and into the region of skin 26 affected by electrodes 32 and 34. Alternatively or additionally, a non-explosive rapid expansion of gas is used to create a force to drive plunger 22 (not shown) to compress the substance and thus push it through the outlet. Further alternatively or additionally, the expanding gas directly compresses the substance or the reservoir, in order to expel the substance from device 70. Expanding gas can be generated inexpensively and safely by reacting, for example, citric acid and sodium bicarbonate. Still further alternatively or additionally, an electrolytic process is used to generate the gas, either to move the plunger or to directly drive the substance towards the outlet. In these examples of embodiments using expanding gas, some or all of the techniques and apparatus described hereinabove with reference to FIGS. 1A, 1B, and 2-4 are optionally used to further enhance the desired transdermal movement of the substance.

FIG. 6 is a schematic sectional illustration of an additional device 80 for transdermal delivery of substance 28, in accordance with a preferred embodiment of the present invention. Device 80 comprises a pump 88, preferably a two-chamber pump, which generates pressure to expel substance 28 from a reservoir 86 into an electrode compartment 90. On a lower surface of compartment 90, an array of electrodes 82 is preferably disposed in the proximity of a plurality of outlets 84. The substance preferably passes into skin 26 through the outlets, responsive to the pressure generated by pump 88 and to current injected into skin 26 by electrodes 82.

It will be appreciated that the individual preferred embodiments described above are cited by way of example, and that specific applications of the present invention will typically employ features described with reference to a plurality of the figures. The full scope of the invention is limited only by the claims.

1. Apparatus for injection of a substance through the stratum corneum of skin, comprising:
a reservoir, containing the substance, the reservoir having at least one outlet;
a pressure generator, which applies a pressure to eject the substance through the at least one outlet; and
one or more electrodes in a vicinity of the outlet, which are placed on the skin, through which electrodes energy is conveyed to the skin to facilitate passage of the substance through the stratum corneum.

2. Apparatus according to claim 1, wherein the one or more electrodes comprise an array of two or more electrodes which convey current into the skin.

3. Apparatus according to claim 1, wherein the substance comprises a powder having an electric charge associated therewith, and wherein the pressure generator comprises first and second accelerating electrodes which generate an electric field to accelerate the powder and cause it to pass through the at least one outlet.

4. Apparatus according to claim 1, and comprising a charge-storage element which conveys electrical energy to the electrodes.

5. Apparatus according to claim 1, wherein the at least one outlet comprises a plurality of outlets from which the substance is ejected.

6. Apparatus according to any one of claims 1-5, wherein the electrodes convey electric current into the skin, such that a portion of the stratum corneum is ablated responsive to the current.

7. Apparatus according to claim 6, wherein micro-channels are formed in the skin responsive to the current.

8. Apparatus according to any one of claims 1-5, wherein the pressure comprises pressure generated by an expanding gas.

9. Apparatus according to claim 8, wherein a shock-wave is generated responsive to the expansion of the gas, and wherein the substance is ejected responsive to the shock-wave.

10. Apparatus according to claim 8, wherein an electrolytic reaction causes the gas to expand.

11. Apparatus according to any one of claims 1-5, wherein the reservoir comprises a barrel, and wherein the pressure generator comprises a plunger slidably contained in the barrel, such that compression of the plunger causes the substance to be ejected from the outlet.

12. Apparatus according to claim 11, wherein the plunger is compressed responsive to at least one of: a manually-generated force, a gas-generated force, and a spring-generated force.

13. Apparatus according to claim 11, wherein the electrodes apply electric current to the skin responsive to compression of the plunger.

14. A method for injecting, through the stratum corneum of skin, a substance contained in a reservoir having an outlet, the method comprising:
generating pressure, to eject the substance through the outlet; and
applying electrical energy to the skin, to facilitate passage of the substance through the stratum corneum.

15. A method according to claim 14, wherein applying the energy comprises conveying the energy through an array of two or more electrodes.

16. A method according to claim 14, wherein the substance comprises a powder having an electric charge associated therewith, and wherein generating pressure comprises generating an electric field to accelerate the powder through the outlet.

17. A method according to claim 14, wherein the outlet comprises a plurality of outlets, and wherein generating pressure comprises ejecting the substance from the outlets.

18. A method according to any one of claims 14-17, wherein applying the energy comprises conveying electric current into the skin, such that a portion of the stratum corneum is ablated responsive to the current.

19. A method according to claim 18, wherein conveying current comprises forming micro-channels in the skin.

20. A method according to any one of claims 14-17, wherein generating pressure comprises causing a gas to expand.

21. A method according to claim 20, wherein causing the gas to expand comprises generating a shock-wave, and wherein the substance is ejected responsive to the shock-wave.

22. A method according to claim 20, wherein causing the gas to expand comprises initiating an electrolytic reaction.

23. A method according to any one of claims 14-17, wherein generating pressure comprises compressing a plunger into a barrel containing the reservoir.

24. A method according to claim 23, wherein compressing the plunger comprises generating at least one of: a manually-generated force, a gas-generated force, and a spring-generated force.

25. A method according to claim 23, wherein compressing the plunger comprises actuating the electrodes to convey electric current into the skin.
